(19) United States
(12) Patent Application Publication
(10) Pub. No.: US 2023/0277758 A1
(43) Pub. Date: Sep. 7, 2023

(54) INFUSION CARTRIDGE, INFUSION PUMP, CAP MEMBER, AND FLOW PATH CLOSURE ASSEMBLY

(71) Applicant: TERUMO KABUSHIKI KAISHA, Tokyo (JP)

(72) Inventor: Koshi SANO, Yamanashi (JP)

(73) Assignee: TERUMO KABUSHIKI KAISHA, Tokyo (JP)

(21) Appl. No.: 18/115,714

(22) Filed: Feb. 28, 2023

(30) Foreign Application Priority Data
Mar. 1, 2022 (JP) 2022-031265

(51) Int. Cl. A61M 5/14; A61M 5/142; A61M 39/20

(52) U.S. Cl. CPC A61M 5/1413; A61M 5/142; A61M 39/20

(57) ABSTRACT

An infusion cartridge includes: a cartridge main body that defines a housing space configured to house an infusion solution; a tube member having a proximal end connected to the cartridge main body and defining a flow path communicating with the housing space; and a cap member attachable to and detachable from a distal end of the tube member. The cartridge main body includes a flow path closing portion configured to be changed between (i) a closing position in which the flow path closing portion closes the flow path of the tube member and (ii) an opening position in which the flow path closing portion does not close the flow path of the tube member. The cap member is attachable to the cartridge main body so as to maintain the flow path closing portion in one position among the closing position and the opening position.

### CROSS-REFERENCE TO RELATED APPLICATIONS

This application claims priority to Japanese Application No. 2022-031265, filed on Mar. 1, 2022, the contents of which are incorporated by reference in their entirety.

### BACKGROUND

The present disclosure relates to an infusion cartridge, an infusion pump, a cap member, and a flow path closing assembly.

Conventionally, a portable infusion pump that delivers an infusion solution such as a medicinal solution has been known. The portable infusion pump is used in a state in which an infusion cartridge containing an infusion solution is attached to a pump main body. The portable infusion pump delivers an infusion solution contained in an infusion cartridge through a tube member and administers the infusion solution into a living body of a patient while controlling a flow rate of the infusion solution.

In a portable infusion pump, it is required to have an anti-free flow (AFF) function in order to prevent a large amount of an infusion solution from being erroneously delivered through a tube member when an infusion cartridge is detached from a pump main body. The AFF function is a function of closing the flow path of the tube member to prevent the infusion solution from being delivered when the infusion cartridge is detached from the pump main body in the portable infusion pump. For example, Japanese Patent No. 6222750 discloses an infusion pump that achieves an AFF function by a flow stop assembly.

### SUMMARY

The infusion cartridge is filled with the infusion solution in a state in which the infusion cartridge is detached from the pump main body. At the time of filling the infusion solution, the necessity of closing the flow path of the tube member varies depending on the configuration of the infusion cartridge. That is, it may be necessary or preferable that the flow path of the tube member be closed by the AFF function at the time of filling the infusion solution. Conversely, at the time of filling the infusion solution, it may be necessary or preferable that the flow path of the tube member is not closed by the AFF function. Therefore, there is a demand for measures to prevent the user from setting the operating state of the AFF function to an erroneous or unfavorable state at the time of filling the infusion solution.

An object of the present disclosure is to provide an infusion cartridge, an infusion pump, a cap member, and a flow path closing assembly capable of guiding a user so that an operating state of an AFF function becomes a desired state at the time of filling an infusion solution.

An infusion cartridge according to a first aspect of the present disclosure includes: a cartridge main body that defines a housing space capable of housing an infusion solution; a tube member having a proximal end connected to the cartridge main body and defining a flow path communicating with the housing space; and a cap member attachable to and detachable from a distal end of the tube member, in which the cartridge main body includes a flow path closing portion capable of changing its position between a closing position of closing the flow path of the tube member and an opening position of not closing the flow path of the tube member, the cap member is attachable to the cartridge main body so as to maintain the flow path closing portion in one position of the closing position and the opening position, and the flow path closing portion changes its position from the one position of the closing position and the opening position to another position in conjunction with an operation of detaching the cap member from the cartridge main body.

According to one embodiment of the present disclosure, the flow path closing portion includes: a tube receiving portion that receives a side surface of the tube member; and a tube pressing portion movable relative to the tube receiving portion between a closing position where the tube member is sandwiched between the tube pressing portion and the tube receiving portion to close the flow path and an opening position where the tube pressing portion is separated from the tube receiving portion with respect to the closing position, and the flow path closing portion changes its position between the closing position and the opening position as the tube pressing portion moves relative to the tube receiving portion between the closing position and the opening position.

According to one embodiment of the present disclosure, the tube pressing portion is connected to the tube receiving portion via a hinge portion, and is configured to be rotatable with respect to the tube receiving portion with the hinge portion as an axis.

According to one embodiment of the present disclosure, the cartridge main body includes a biasing portion that biases the tube pressing portion.

As one embodiment of the present disclosure, the tube member includes: a tube main body; and a connector attached to a distal end of the tube main body and configured to be attachable to and detachable from the cap member, and the infusion cartridge further includes an operation restricting portion that restricts a linear pull-out operation of the connector from the cap member in a state in which the cap member is attached to the cartridge main body.

According to one embodiment of the present disclosure, the connector of the tube member is configured to be attachable to and detachable from the cap member by being moved in a linear attachment/detachment direction with respect to the cap member, the cartridge main body includes a restricting wall portion that restricts a movement of the connector attached to the cap member in the attachment/detachment direction by abutting against the tube main body or the connector in a state in which the cap member is attached to the cartridge main body, and the operation restricting portion is the restricting wall portion.

According to one embodiment of the present disclosure, the cartridge main body includes a tube receiving surface that supports the flow path closing portion and receives the tube member on an upstream side and a downstream side of the flow path with respect to the flow path closing portion, the cap member is attachable to the tube receiving surface of the cartridge main body so as to cover the flow path closing portion, and the restricting wall portion is formed by the tube receiving surface.

According to one embodiment of the present disclosure, the cartridge main body includes a filling port capable of filling the housing space with the infusion solution, the cap member is attachable to the cartridge main body so as to maintain the flow path closing portion in the opening position, and the flow path closing portion changes its position from the opening position to the closing position in conjunction with an operation of detaching the cap member from the cartridge main body.

According to infusion pump as a second aspect of the present disclosure includes the infusion cartridge and a pump main body to which the infusion cartridge is attachable.

A cap member according to a third aspect of the present disclosure is a cap member attachable to and detachable from a distal end of a tube member having a proximal end connected to a cartridge main body that defines a housing space capable of housing an infusion solution, in which the cartridge main body includes a flow path closing portion capable of changing its position between a closing position of closing a flow path of the tube member and an opening position of not closing the flow path of the tube member, the cap member is configured to be attachable to the cartridge main body so as to maintain the flow path closing portion in one position of the closing position and the opening position, and the cap member changes a position of the flow path closing portion from the one position of the closing position and the opening position to another position by being detached from the cartridge main body.

A flow path closing assembly according to a fourth aspect of the present disclosure includes: a cap member attachable to and detachable from a distal end of a tube member having a proximal end connected to a cartridge main body defining a housing space capable of housing an infusion solution; and a flow path closing member attachable to the cartridge main body and capable of changing its position between a closing position of closing a flow path of the tube member and an opening position of not closing the flow path of the tube member, in which the cap member is configured to be attachable to the flow path closing member so as to maintain the flow path closing portion in one position of the closing position and the opening position, and the cap member changes a position of the flow path closing member from the one position of the closing position and the opening position to another position by being detached from the flow path closing member.

According to certain embodiments the present disclosure, it is possible to provide an infusion cartridge, an infusion pump, a cap member, and a flow path closing assembly capable of guiding a user so that an operating state of an AFF function becomes a desired state at the time of filling an infusion solution.

### BRIEF DESCRIPTION OF THE DRAWINGS

FIG. 1 is a front view illustrating an infusion pump as an embodiment of the present disclosure;

FIG. 2A is a view illustrating a state immediately before an infusion cartridge illustrated in FIG. 1 is attached to a pump main body illustrated in FIG. 1;

FIG. 2B is a view illustrating a state in which the infusion cartridge illustrated in FIG. 1 is being attached to the pump main body illustrated in FIG. 1;

FIG. 2C is a view illustrating a state in which attachment of the infusion cartridge illustrated in FIG. 1 to the pump main body illustrated in FIG. 1 is completed;

FIG. 3 is a cross-sectional view of a single infusion cartridge illustrated in FIG. 1, and is a view illustrating a state in which an infusion solution is not housed in a housing space of the infusion cartridge;

FIG. 4 is a cross-sectional view of the single infusion cartridge illustrated in FIG. 1, and is a view illustrating a state in which an infusion solution is housed in a housing space of the infusion cartridge;

FIG. 5A is a perspective view of the single infusion cartridge illustrated in FIG. 1, and is a view illustrating a state in which a cap member is attached to a cartridge main body;

FIG. 5B is a perspective view of the single infusion cartridge illustrated in FIG. 1, and is a view illustrating a state in which the cap member is detached from the cartridge main body;

FIG. 6 is a perspective view illustrating a single flow path closing member constituting a flow path closing portion illustrated in FIG. 5B;

FIG. 7A is a perspective view of a single cap member illustrated in FIG. 1;

FIG. 7B is a perspective view of the single cap member as viewed from a position different from that in FIG. 7A;

FIG. 7C is a side view of the single cap member illustrated in FIG. 1;

FIG. 8 is a perspective view of a connector of a tube member illustrated in FIG. 1 attachable to and detachable from the cap member illustrated in FIG. 1;

FIG. 9 is an explanatory view for explaining an attachment configuration of a tube receiving surface of a container illustrated in FIGS. 5A and 5B, a flow path closing member illustrated in FIG. 6, and a cap member illustrated in FIG. 1;

FIG. 10 is a perspective view illustrating details of a state in which the cap member illustrated in FIG. 1 is attached to the cartridge main body illustrated in FIG. 1;

FIG. 11 is a cross-sectional view taken along line I-I in FIG. 10;

FIG. 12 is a perspective view illustrating details of a state in which the cap member illustrated in FIG. 10 is detached from the cartridge main body;

FIG. 13 is a cross-sectional view taken along line II-II in FIG. 12; and

FIG. 14 is a view illustrating an infusion cartridge as a modification of the infusion cartridge illustrated in FIG. 1.

### DETAILED DESCRIPTION

Hereinafter, embodiments of an infusion cartridge, an infusion pump, a cap member, and a flow path closing assembly according to the present disclosure will be described by way of example with reference to the drawings. In the drawings, the same components are denoted by the same reference numerals.

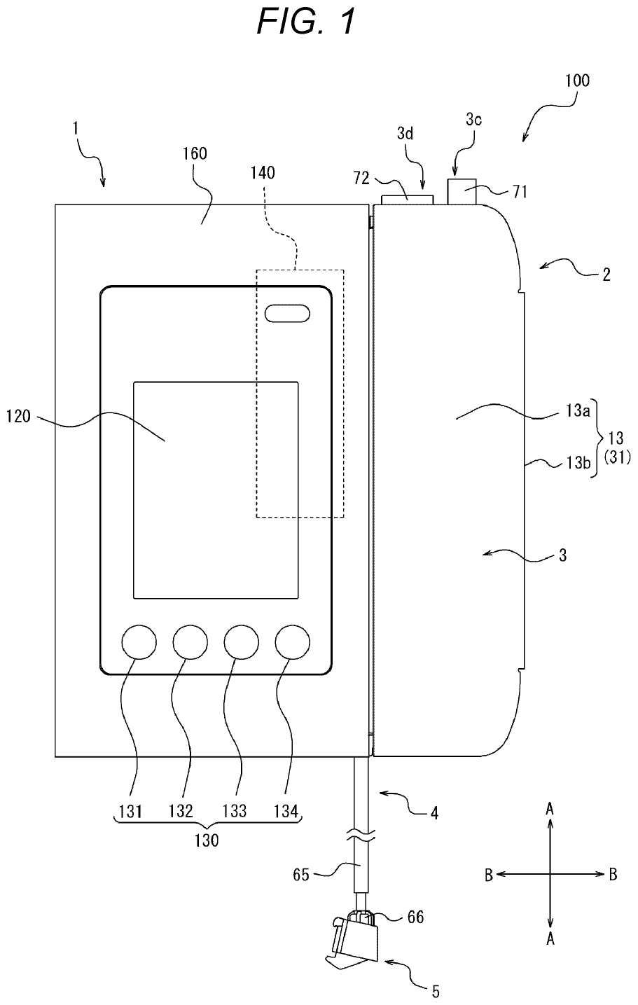

FIG. 1 is a front view of an infusion pump 100 as an embodiment of an infusion pump according to the present disclosure. As illustrated in FIG. 1, the infusion pump 100 includes a pump main body 1 and an infusion cartridge 2 as an embodiment of an infusion cartridge according to the present disclosure. The infusion pump 100 illustrated in FIG. 1 can be used as, for example, a PCA (Patient Controlled Analgesia) pump, but the application thereof is not particularly limited. In the infusion pump 100 of the present embodiment, the pump main body 1 can be reused by replacing the disposable infusion cartridge 2.

<Pump Main Body 1>

As illustrated in FIG. 1, a display unit 120 on which various types of information are displayed and an operation unit 130 in which operation switches and the like are arranged are arranged on the front surface of the pump main body 1. The display unit 120 displays, for example, a liquid delivery rate, an integrated dose, and the like. Furthermore, the display unit 120 may be a liquid crystal screen with a touch panel for setting a liquid delivery rate and the like. The operation switch of the operation unit 130 illustrated in FIG. 1 includes a fast delivery switch 131, a start switch 132, a stop switch 133, and a power switch 134. While the fast delivery switch 131 is pressed, liquid can be delivered at a liquid delivery rate higher than the set liquid delivery rate (mL/h). When the start switch 132 is pressed, liquid delivery can be started. When the stop switch 133 is pressed, the liquid delivery can be forcibly stopped. When the power switch 134 is pressed, ON/OFF of the power supply of the pump main body 1 can be switched. However, the operation unit 130 may include another operation switch. The display unit 120 and the operation unit 130 make it possible to deliver an infusion solution such as a medicinal solution into a living body of a patient or the like while controlling a liquid delivery amount.

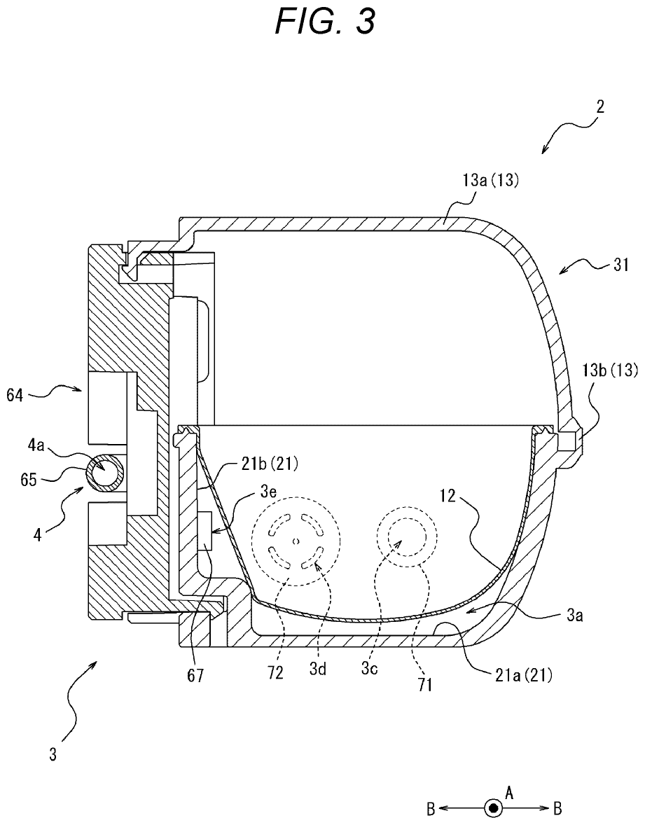

In addition, the pump main body 1 includes a liquid delivery unit 140 that sandwiches a tube member 4 of the infusion cartridge 2 between the pump main body 1 and a tube receiving surface 64 (see FIGS. 3 to 5B and the like) to be described later of the infusion cartridge 2 to be attached and delivers the infusion solution in the tube member 4 from the flow path upstream side to the flow path downstream side. The liquid delivery unit 140 of the present embodiment includes a plurality of fingers and a drive unit that drives the fingers. The plurality of fingers are arranged on a facing surface of the pump main body 1 facing the tube receiving surface 64 (see FIGS. 3 to 5B and the like) to be described later located on a side surface of the infusion cartridge 2. The plurality of fingers are arranged along an extending direction A of the tube member 4. Each finger is driven by the drive unit so as to reciprocate in an opposing direction B with respect to the tube receiving surface 64 (see FIGS. 3 to 5B and the like) to be described later of the infusion cartridge 2. As each finger moves close to the infusion cartridge 2, the tube member 4 is sandwiched between each finger and the tube receiving surface 64. Accordingly, the tube member 4 is compressed and closed. The drive unit sequentially drives the fingers from the flow path upstream side toward the flow path downstream side in the extending direction A of the tube member 4. The drive unit may include, for example, an electric motor driven by a power source such as a battery, a drive mechanism that operates the finger, and the like. As a result, the tube member 4 is sequentially compressed and closed from the flow path upstream side toward the flow path downstream side, and peristaltically moves. Therefore, the infusion solution in the tube member 4 can be delivered from the flow path upstream side toward the flow path downstream side.

In addition, the pump main body 1 of the present embodiment includes an exterior member 160. That is, the outer surface of the pump main body 1 of the present embodiment is covered with the exterior member 160. The display unit 120 and the operation unit 130 described above are exposed to the outside from an opening positioned in the exterior member 160. The exterior member 160 may be formed of, for example, various resin materials. For example, the exterior member 160 may be integrally formed of a single member, or may be formed of two or more members.

The pump main body 1 is not limited to the configuration of the present embodiment. The pump main body 1 may include a portion different from the above-described portion, for example, a notification unit such as a bubble detection sensor unit or a blockage sensor unit. Furthermore, as described above, the liquid delivery unit 140 of the pump main body 1 of the present embodiment is configured to press the tube member 4 by the plurality of fingers, but may include a pressing unit different from the fingers as long as it is configured to be able to deliver the infusion solution in the tube member 4.

<Infusion Cartridge 2>

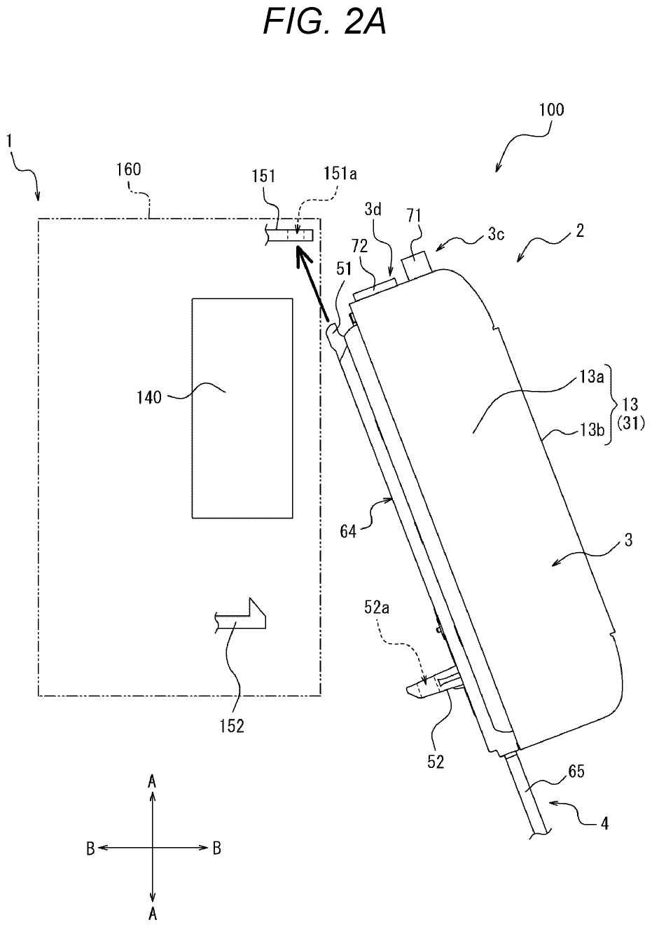

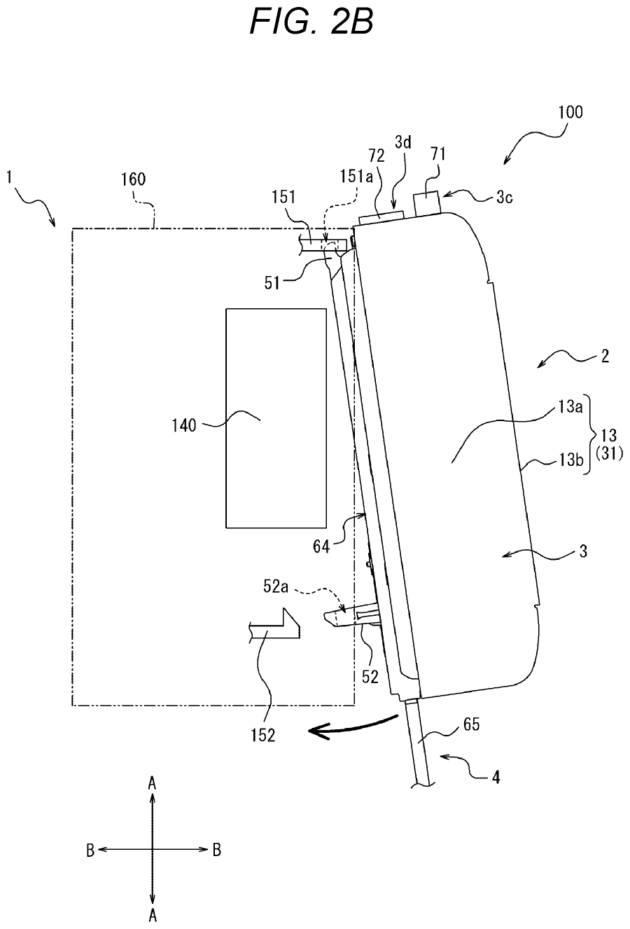

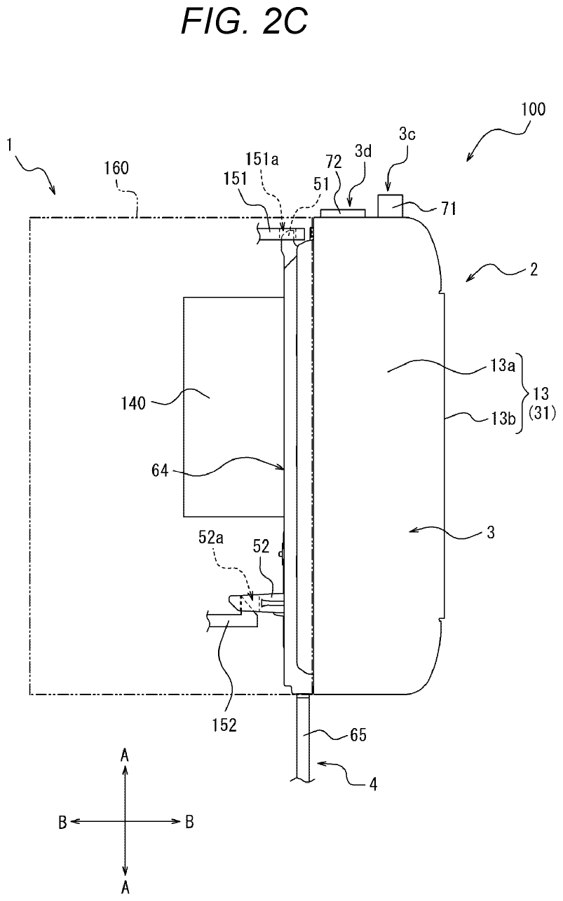

The infusion cartridge 2 can be attached to the pump main body 1. First, an attachment operation of the infusion cartridge 2 to the pump main body 1 will be described with reference to FIGS. 2A to 2C. FIG. 2A illustrates a state immediately before the infusion cartridge 2 is attached to the pump main body 1. FIG. 2B illustrates a state in the middle of attaching the infusion cartridge 2 to the pump main body 1. FIG. 2C illustrates an attachment state in which attachment of the infusion cartridge 2 to the pump main body 1 is completed. As illustrated in FIGS. 2A to 2C, the pump main body 1 of the present embodiment includes a receiving portion 151 in which a through hole 151a is formed, and a locking claw portion 152. In addition, the infusion cartridge 2 of the present embodiment includes a swing shaft portion 51 and a claw receiving portion 52 in which a through hole 52a is formed.

As illustrated in FIG. 2A, when the infusion cartridge 2 is attached to the pump main body 1, the swing shaft portion 51 of the infusion cartridge 2 is fitted into the through hole 151a of the receiving portion 151 of the pump main body 1. Next, as illustrated in FIG. 2B, the infusion cartridge 2 is swung around the swing shaft portion 51 in a state in which the swing shaft portion 51 is fitted in the through hole 151a. As a result, as illustrated in FIG. 2C, the locking claw portion 152 of the pump main body 1 enters the through hole 52a of the claw receiving portion 52 of the infusion cartridge 2 to lock the claw receiving portion 52. As a result, the attachment of the infusion cartridge 2 to the pump main body 1 is completed. More specifically, in the present embodiment, the attachment of the infusion cartridge 2 to the pump main body 1 is completed when the swing shaft portion 51 of the infusion cartridge 2 is fitted into the through hole 151a of the receiving portion 151 of the pump main body 1 and the locking claw portion 152 of the pump main body 1 enters the through hole 52a of the claw receiving portion 52 of the infusion cartridge 2.

In addition, when the infusion cartridge 2 attached to the pump main body 1 is detached from the pump main body 1, an operation opposite to the attachment operation described above may be executed. First, the locking claw portion 152 of the pump main body 1 is removed from the through hole 52a of the claw receiving portion 52 of the infusion cartridge 2. Next, the infusion cartridge 2 is swung in a direction opposite to the arrow direction in FIG. 2B. Next, the swing shaft portion 51 of the infusion cartridge 2 is removed from the through hole 151a of the receiving portion 151 of the pump main body 1. As a result, the detaching of the infusion cartridge 2 from the pump main body 1 is completed.

In the present embodiment, attachment and detachment of the pump main body 1 and the infusion cartridge 2 are realized by the receiving portion 151 and the locking claw portion 152 of the pump main body 1 and the swing shaft portion 51 and the claw receiving portion 52 of the infusion cartridge 2, but a configuration for realizing attachment and detachment of the pump main body 1 and the infusion cartridge 2 is not particularly limited. Therefore, the pump main body 1 and the infusion cartridge 2 may be detachable using a mechanism different from the present embodiment.

Next, details of the infusion cartridge 2 will be described with reference to FIGS. 3 to 13.

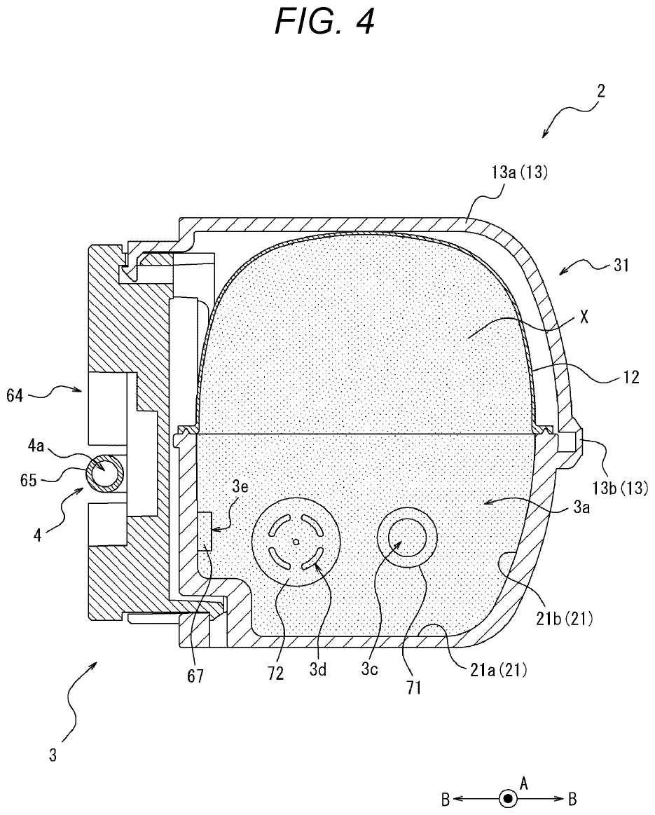

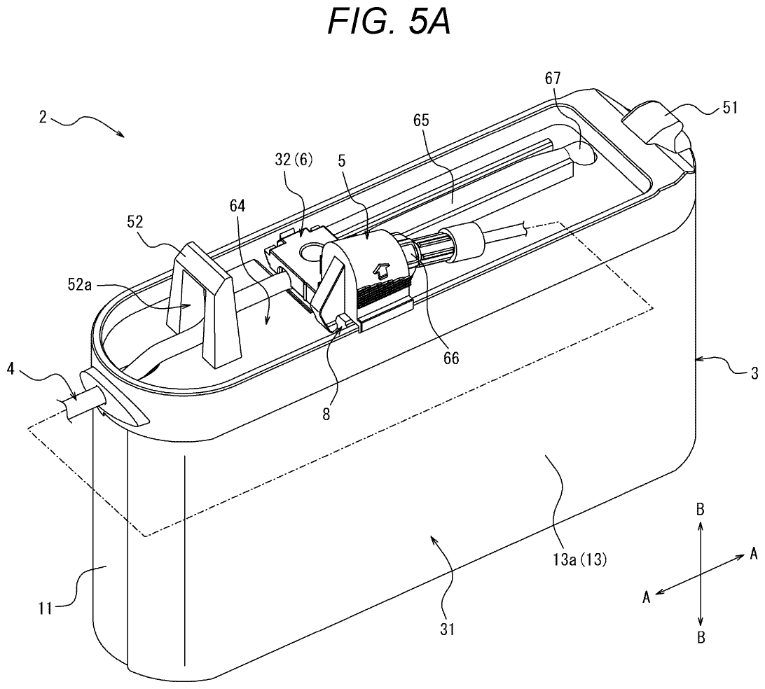

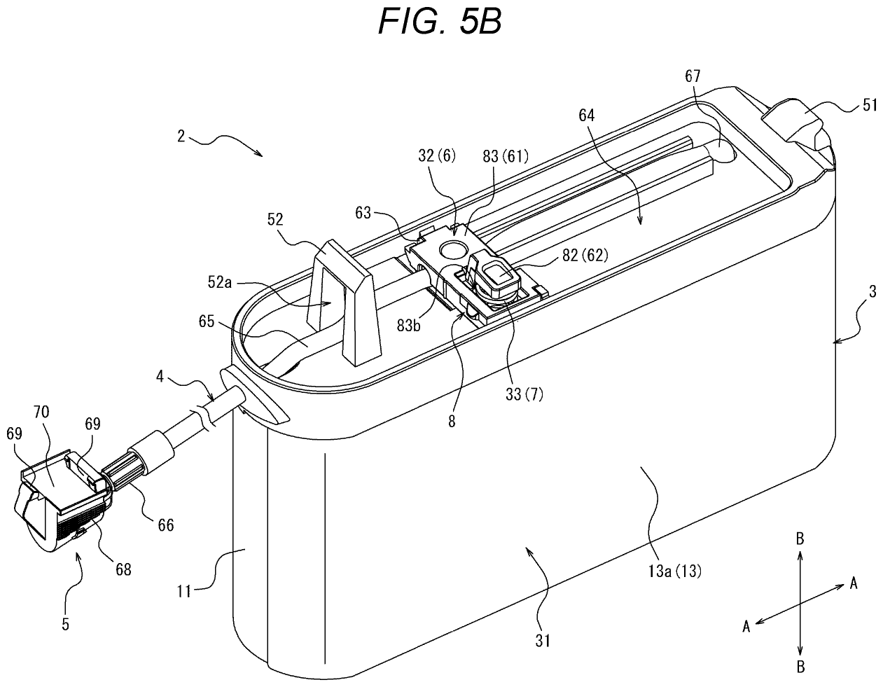

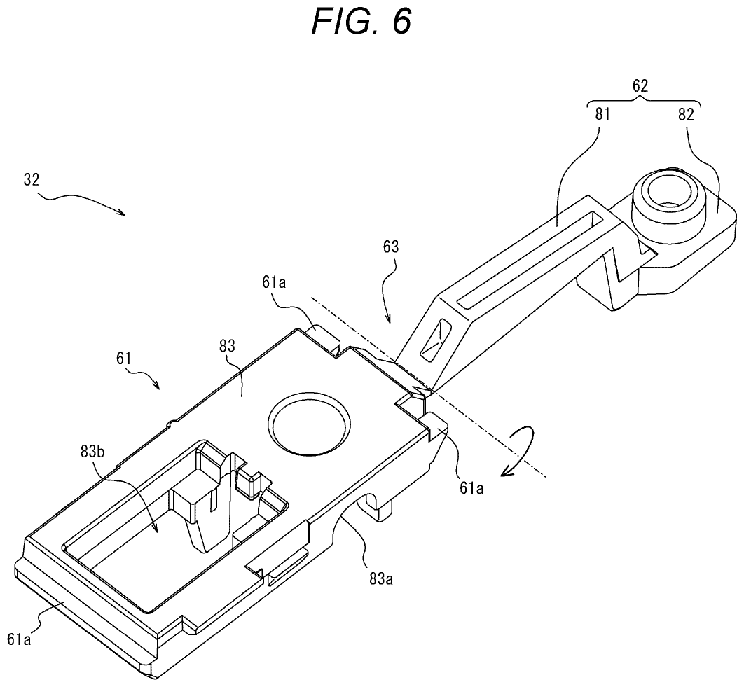

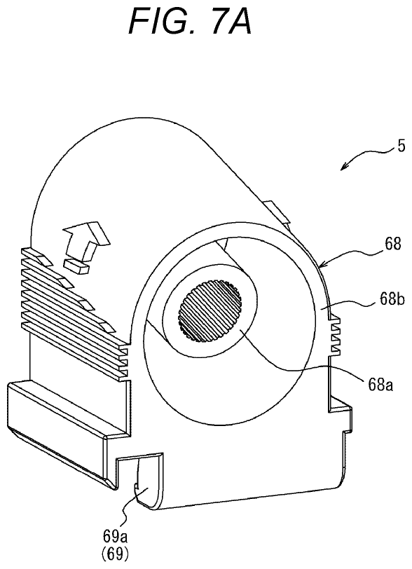

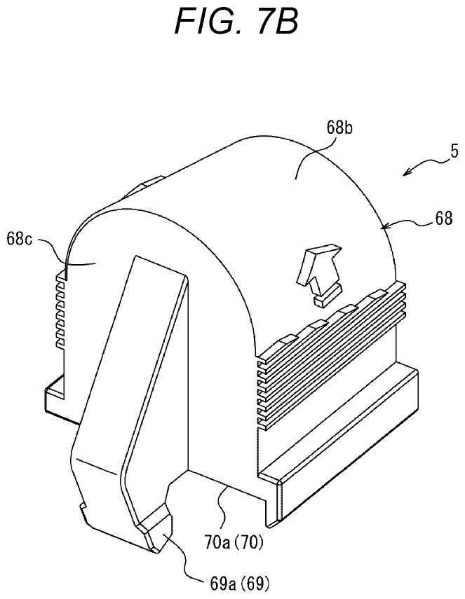

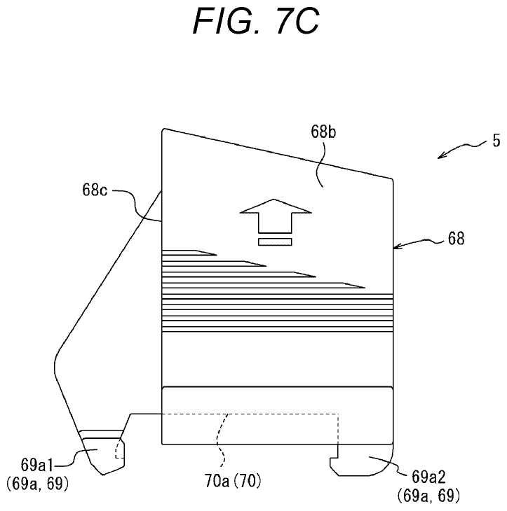

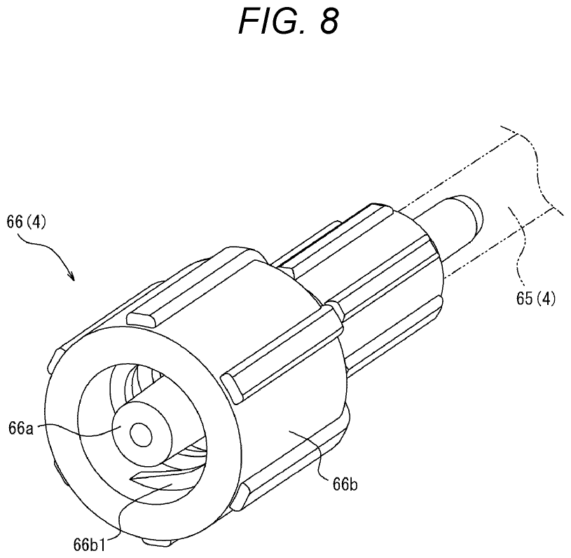

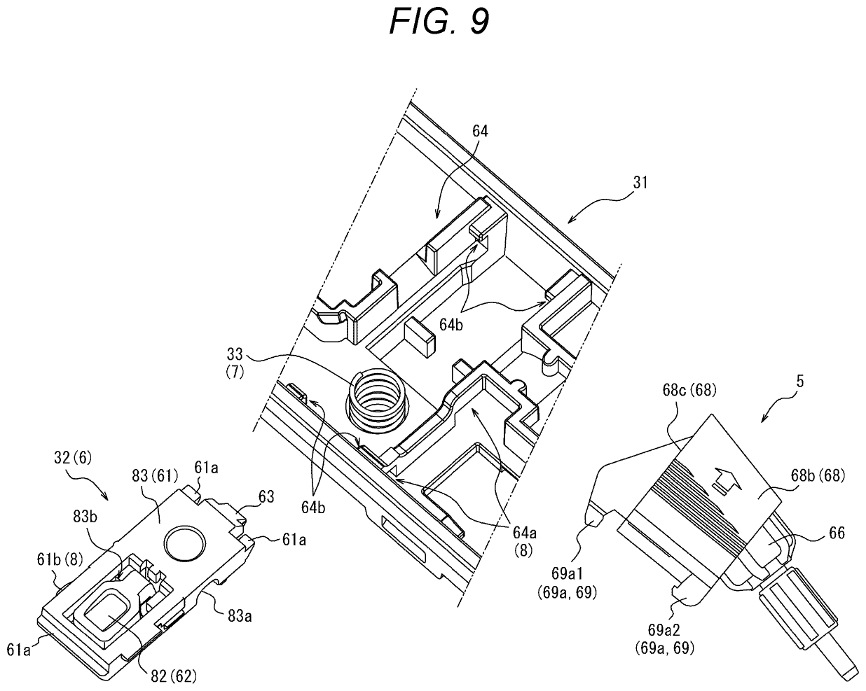

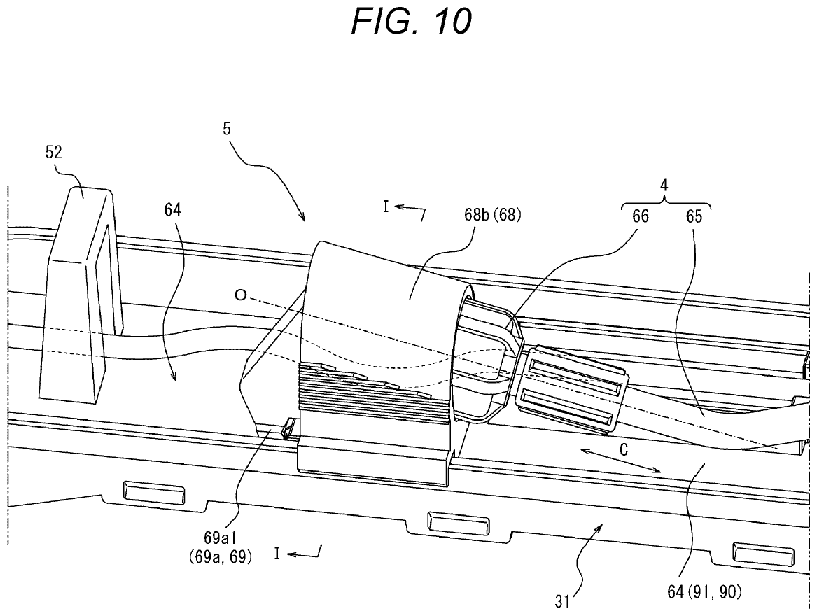

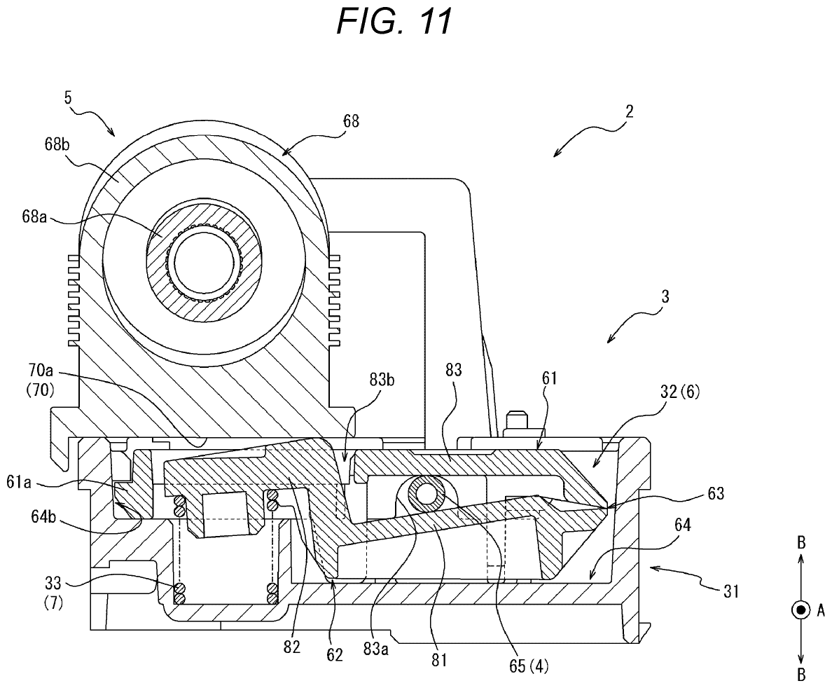

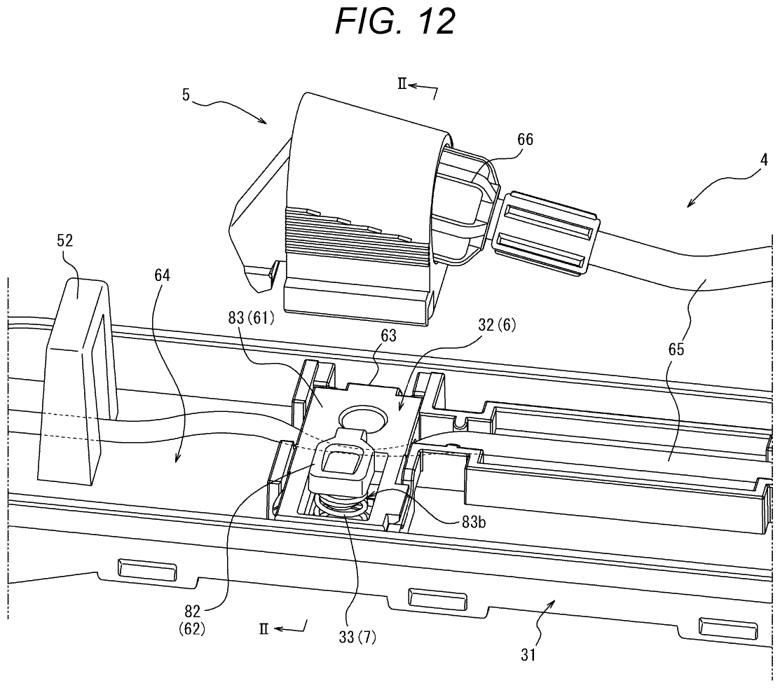

FIGS. 3 and 4 are cross-sectional views of the single infusion cartridge 2. FIG. 3 illustrates a state in which an infusion solution X is not housed in a housing space 3a. FIG. 4 illustrates a state in which the infusion solution X is housed in the housing space 3a. FIGS. 5A and 5B are perspective views of the single infusion cartridge 2. FIG. 5A illustrates a state in which a cap member 5 is attached to the cartridge main body 3. FIG. 5B illustrates a state in which the cap member 5 is detached from the cartridge main body 3. FIG. 6 is a perspective view illustrating a single flow path closing member 32 constituting a flow path closing portion 6. FIGS. 7A and 7B are perspective views of the single cap member 5 as viewed from different positions. FIG. 7C is a side view of the single cap member 5. FIG. 8 is a perspective view of a connector 66 of the tube member 4. FIG. 9 is an explanatory view for explaining an attachment configuration of the tube receiving surface 64 of a container 31, the flow path closing member 32, and the cap member 5. FIG. 10 is a perspective view illustrating details of a state in which the cap member 5 is attached to the cartridge main body 3. FIG. 11 is a cross-sectional view at a position on line I-I in FIG. 10. FIG. 12 is a perspective view illustrating details of a state in which the cap member 5 is removed from the cartridge main body 3.

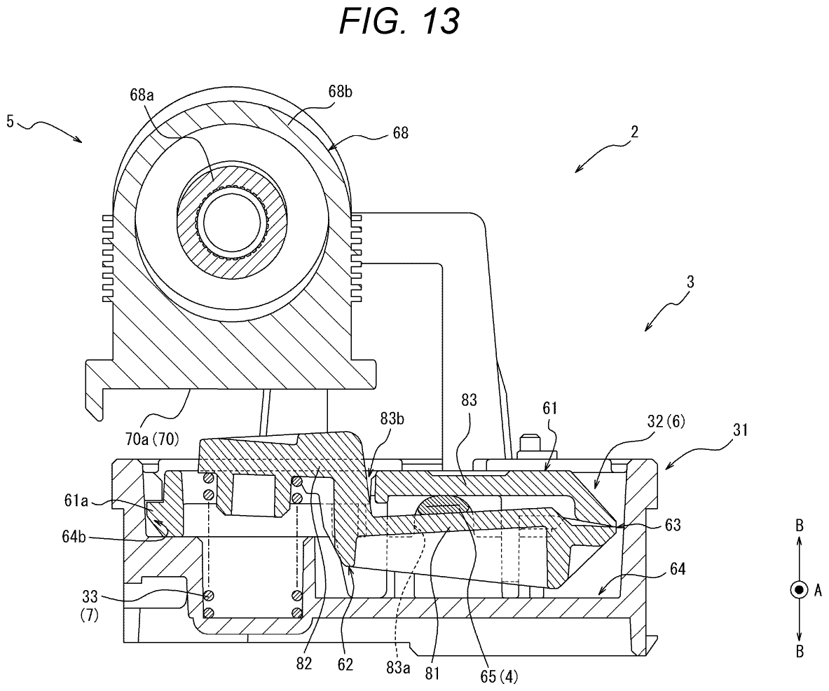

FIG. 13 is a cross-sectional view taken along line II-II in FIG. 12. However, for convenience of explanation, in FIGS. 11 and 13, the connector 66 attached to the cap member 5 is not illustrated.

As illustrated in FIGS. 5A, 5B, and the like, the infusion cartridge 2 includes a cartridge main body 3, a tube member 4, and a cap member 5.

As illustrated in FIGS. 3, 4, and the like, the cartridge main body 3 defines a housing space 3a capable of housing the infusion solution X. A filling port 3c and a ventilation port 3d are formed on an inner wall defining the housing space 3a. The housing space 3a can be filled with the infusion solution X from the outside through the filling port 3c. The gas in the housing space 3a can be discharged to the outside through the ventilation port 3d. In addition, as illustrated in FIGS. 5A, 5B, and the like, a proximal end of the tube member 4 is connected to the cartridge main body 3. The tube member 4 defines a flow path 4a. The flow path 4a of the tube member 4 communicates with the housing space 3a. The infusion solution X housed in the housing space 3a is administered to the living body through the flow path 4a of the tube member 4. The cap member 5 is configured to be detachable from a distal end of the tube member 4. FIGS. 5A and 5B illustrate a state in which the cap member 5 is attached to the distal end of the tube member 4.

As illustrated in FIGS. 5A and 5B, the cartridge main body 3 includes a flow path closing portion 6. The flow path closing portion 6 can change its position between a closing position and an opening position. The closing position of the flow path closing portion 6 is a position in which the flow path closing portion 6 closes the flow path 4a of the tube member 4. The opening position of the flow path closing portion 6 is a position in which the flow path closing portion 6 does not close the flow path 4a of the tube member 4. The detailed configuration and operation of the flow path closing portion 6 of the present embodiment will be described later (See FIG. 11, FIG. 13, etc.).

The cap member 5 of the present embodiment is configured to be attachable to the cartridge main body 3 so as to maintain the flow path closing portion 6 in the opening position. FIG. 5A illustrates the cap member 5 in a state of being attached to the cartridge main body 3 so as to maintain the flow path closing portion 6 in the opening position. This will be described later in detail (See FIGS. 10 and 11).

The flow path closing portion 6 of the present embodiment changes its position from the opening position to the closing position in conjunction with the operation of detaching the cap member 5 from the cartridge main body 3. FIG. 5B illustrates a state in which the cap member 5 illustrated in FIG. 5A is detached from the cartridge main body 3. That is, the flow path closing portion 6 shown in FIG. 5B shows a state after the position is changed from the opening position to the closing position. This will be described later in detail (See FIGS. 12 and 13).

As described above, in the infusion cartridge 2, the position change of the closing position and the opening position of the flow path closing portion 6 can be executed by the attaching and detaching operation of the cap member 5 attached to the distal end of the tube member 4 to and from the cartridge main body 3.

Here, the infusion cartridge 2 of the present embodiment is packaged and distributed in a state in which the cap member 5 is attached to the distal end of the tube member 4. That is, in the infusion cartridge 2 of the present embodiment, the cap member 5 is attached to the distal end of the tube member 4 when the infusion cartridge 2 is taken out from the packaging container for use.

In addition, the infusion cartridge 2 of the present embodiment is packaged and distributed in a state in which the cap member 5 is attached to the cartridge main body 3 (see FIG. 5A). That is, in the infusion cartridge 2 of the present embodiment, the cap member 5 is attached to the cartridge main body 3 when the infusion cartridge 2 is taken out from the packaging container for use.

Although details will be described later, in the infusion cartridge 2 of the present embodiment, the flow path 4a of the tube member 4 is preferably closed when the infusion solution X (see FIG. 4) is filled. On the other hand, in consideration of sterilization treatment at the time of manufacture and plastic deformation of the tube member 4 from manufacture to use, in the infusion cartridge 2 of the present embodiment, it is preferable that the flow path 4a of the tube member 4 is not closed until the time of use of filling the housing space 3a with the infusion solution X. Therefore, in the infusion cartridge 2 of the present embodiment, at the time of use in which the infusion cartridge 2 is taken out from the packaging container and the housing space 3a is filled with the infusion solution X (see FIG. 4), it is preferable that the flow path 4a of the tube member 4 is switched from the unclosed state to the closed state.

Therefore, the cap member 5 of the present embodiment is attached to the cartridge main body 3 in advance so that the flow path closing portion 6 maintains the opening position (see FIG. 5A). The user normally grips the distal end of the tube member 4 so as to face a predetermined direction (for example, upward in the vertical direction) in order to suppress the infusion solution X filled in the housing space 3a from leaking from the distal end of the tube member 4 through a path (filling port 3c in the present embodiment) different from the flow path 4a of the tube member 4. That is, when the cap member 5 attached to the distal end of the tube member 4 is attached to the cartridge main body 3 when the infusion cartridge 2 is taken out from the packaging container, the user tends to execute an operation of detaching the cap member 5 from the cartridge main body 3 in order to direct the distal end of the tube member 4 in a predetermined direction. When the user detaches the cap member 5 from the cartridge main body 3, the position of the flow path closing portion 6 changes from the opening position to the closing position. As a result, the flow path closing portion 6 of the present embodiment becomes a preferable position at the time of filling the infusion solution X into the infusion cartridge 2.

That is, according to the infusion cartridge 2 of the present embodiment, by using the cap member 5, it is possible to guide the user so that the operating state of the AFF function becomes a desired state when the infusion cartridge 2 is filled with the infusion solution X (see FIG. 4).

In particular, as illustrated in FIG. 5A, the cap member 5 of the present embodiment is attached so as to cover the tube receiving surface 64 of the cartridge main body 3 that sandwiches the tube member 4 between the pump main body 1 and the tube receiving surface 64. The infusion cartridge 2 of the present embodiment cannot be attached to the pump main body 1 in a state in which the cap member 5 is attached so as to cover the tube receiving surface 64 of the cartridge main body 3 (see FIGS. 2A to 2C). In other words, the cap member 5 of the present embodiment is attached to the cartridge main body 3 at a position that becomes an obstacle when the infusion cartridge 2 is attached to the pump main body 1. Therefore, the cap member 5 of the present embodiment is more easily detached from the cartridge main body 3 by the user at the time of use.

As described above, the cap member 5 of the present embodiment is configured to be attachable to the cartridge main body 3 so as to maintain the flow path closing portion 6 in the opening position, but is not limited to this configuration. The cap member 5 may be configured to be attachable to the cartridge main body 3 so as to maintain the flow path closing portion 6 in the closing position. In the case of such a cap member 5, the flow path closing portion 6 changes its position from the closing position to the opening position in conjunction with the operation of detaching the cap member 5 from the cartridge main body 3.

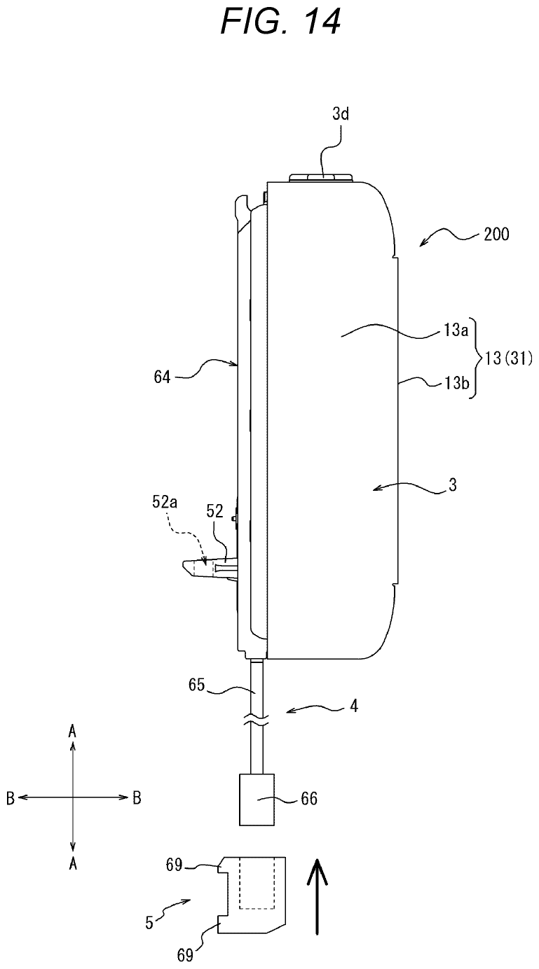

Although details will be described later, there is an infusion cartridge in which the flow path 4a of the tube member 4 is preferably opened when the infusion solution X (see FIG. 4) is filled. Examples of this type of infusion cartridge include an infusion cartridge in which the housing space 3a is filled with the infusion solution X through the flow path 4a of the tube member 4 (see FIG. 14). Even in this type of infusion cartridge, as described above, in consideration of the sterilization treatment at the time of manufacture and the plastic deformation of the tube member 4 from manufacture to use, the flow path 4a of the tube member 4 is preferably not closed before use.

Therefore, in this type of infusion cartridge, the cap member 5 is detached from the cartridge main body 3 so that the flow path closing portion 6 maintains the opening position when the infusion cartridge is detached from the packaging container. Therefore, the user is not guided to the operation of detaching the cap member 5 from the cartridge main body 3. In other words, the user is guided to leave the state in which the cap member 5 is detached from the cartridge main body 3. Therefore, the user can fill the infusion solution X (see FIG. 4) without performing the attaching and detaching operation of the cap member 5 to and from the cartridge main body 3. That is, even in this type of infusion cartridge, it is possible to guide the user so that the operating state of the AFF function becomes a desired state when the infusion cartridge is filled with the infusion solution X (see FIG. 4).

As described above, the cap member 5 may be attachable to the cartridge main body 3 so as to maintain the flow path closing portion 6 in one of the closing position and the opening position. In such a case, the flow path closing portion 6 is configured to change the position from one of the closing position and the opening position to another position in conjunction with the operation of detaching the cap member 5 from the cartridge main body 3. In this manner, it is possible to guide the user so that the operating state of the AFF function becomes a desired state at the time of filling the infusion solution X (see FIG. 4) into the infusion cartridge.

Next, further details of the infusion cartridge 2 of the present embodiment will be described.

As described above, the infusion cartridge 2 includes the cartridge main body 3, the tube member 4, and the cap member 5.

As described above, the cartridge main body 3 defines the housing space 3a capable of housing the infusion solution X (see FIG. 4). In addition, as described above, the cartridge main body 3 of the present embodiment includes the filling port 3c and the ventilation port 3d communicating with the housing space 3a. Furthermore, as illustrated in FIGS. 3 and 4, the cartridge main body 3 includes an administration port 3e that allows the flow path 4a of the tube member 4 to communicate with the housing space 3a.

The filling port 3c of the present embodiment is defined in a cylindrical portion 71. A valve body for suppressing backflow may be disposed in the filling port 3c of the cylindrical portion 71. In addition, on the outer surface of the cylindrical portion 71, for example, a male screw portion to which a locking male connector portion can be screwed may be formed.

The housing space 3a of the cartridge main body 3 of the present embodiment is filled with the infusion solution X through the filling port 3c by a filling tool such as a syringe. In other words, the housing space 3a of the cartridge main body 3 of the present embodiment is not filled with the infusion solution X through the flow path 4a of the tube member 4. Therefore, when the flow path 4a of the tube member 4 is not closed at the time of filling the housing space 3a with the infusion solution X, there is a possibility that a part of the infusion solution X filled in the housing space 3a from the filling port 3c enters the flow path 4a through the administration port 3e. It is not preferable that the infusion solution X unintentionally enters the flow path 4a of the tube member 4 from the viewpoint of the risk of leakage of the infusion solution X from the distal end of the tube member 4 and the viewpoint of management of the filling amount of the infusion solution X to be filled in the housing space 3a. Therefore, in the infusion cartridge 2 of the present embodiment, the flow path 4a of the tube member 4 is preferably closed when the housing space 3a is filled with the infusion solution X.

The ventilation port 3d of the present embodiment is a through hole defined by a ventilation plate portion 72. It is preferable that a ventilation filter is disposed in the ventilation port 3d. Furthermore, as the ventilation filter, it is preferable to use a configuration in which the surface is hydrophobized or a hydrophobic membrane (hydrophobic membrane). Examples of a constituent material of the hydrophobic membrane include polytetrafluoroethylene (PTFE), a copolymer of tetrafluoroethylene and hexafluoropropylene (FEP), a copolymer of tetrafluoroethylene and perfluoroalkyl vinyl ether (PFA), and polyvinylidene fluoride (PVDF). The ventilation filter preferably has a porous structure. The ventilation filter having a porous structure can be obtained, for example, by making the above-described constituent material of the hydrophobic membrane porous by a method such as a stretching method, a micro phase separation method, an electron beam etching method, a sintering method, or argon plasma particles. In addition, the method of the hydrophobizing treatment is not particularly limited, and for example, a method of coating the surface of the ventilation filter with a hydrophobic constituent material or the like may be used. Furthermore, the ventilation filter is not limited to the above configuration as long as it has a hydrophobic configuration having a function of allowing permeation of gas and not allowing permeation of liquid, and may have a configuration including a material of another material such as a hydrophilic material, for example. With such a hydrophobic ventilation filter, the gas in the housing space 3a can be discharged to the outside, and the infusion solution X in the housing space 3a can be suppressed from leaking to the outside.

The cartridge main body 3 of the present embodiment includes a flow path closing portion 6, a biasing portion 7, and a cap attachment portion 8.

The flow path closing portion 6 of the present embodiment includes a tube receiving portion 61 and a tube pressing portion 62. The tube receiving portion 61 receives a side surface of the tube member 4. As illustrated in FIGS. 10 to 13, the tube pressing portion 62 is movable relative to the tube receiving portion 61 between a closing position (see FIG. 13) where the tube member 4 is sandwiched between the tube pressing portion 62 and the tube receiving portion 61 to close the flow path 4a and an opening position (see FIG. 11) where the tube pressing portion 62 is spaced apart from the tube receiving portion 61 from the closing position. As described above, the position of the flow path closing portion 6 can be changed between the closing position and the opening position. In the flow path closing portion 6 of the present embodiment, the tube pressing portion 62 moves relative to the tube receiving portion 61 between the closing position and the opening position, so that the position can be changed between the closing position and the opening position described above.

In addition, the flow path closing portion 6 of the present embodiment includes a hinge portion 63 in addition to the tube receiving portion 61 and the tube pressing portion 62. The tube pressing portion 62 of the present embodiment is connected to the tube receiving portion 61 via the hinge portion 63. The tube pressing portion 62 of the present embodiment is configured to be rotatable with respect to the tube receiving portion 61 with the hinge portion 63 as an axis.

More specifically, as shown in FIGS. 5A, 5B, and 6, the flow path closing portion 6 of the present embodiment is formed by the flow path closing member 32. The flow path closing member 32 is used as the flow path closing portion 6 by being attached to the container 31 described later. Details of the flow path closing member 32 will be described later.

As illustrated in FIGS. 5B and 11 to 13, the biasing portion 7 biases the tube pressing portion 62 of the flow path closing portion 6. The biasing portion 7 of the present embodiment is formed by a biasing member 33. The biasing member 33 of the present embodiment is a coil spring. The biasing portion 7 of the present embodiment biases the tube pressing portion 62 so that the tube pressing portion 62 is at the closing position (see FIG. 13). That is, the biasing portion 7 of the present embodiment biases the tube pressing portion 62 of the flow path closing portion 6 toward the tube receiving portion 61 (see FIGS. 11, 13, and the like). The tube pressing portion 62 of the present embodiment can sandwich the tube member 4 between the tube receiving portion 61 and the tube pressing portion 62 and close the flow path 4a of the tube member 4 by being biased by the biasing portion 7.

The cap attachment portion 8 is configured to be able to attach the cap member 5. The cap attachment portion 8 of the present embodiment is a claw receiving portion on which a locking claw portion 69a (See FIGS. 7A to 7C and FIG. 9) as an attachment portion 69 to be described later of the cap member 5 is caught.

More specifically, the cartridge main body 3 of the present embodiment includes a container 31, a flow path closing member 32, and a biasing member 33. In the present embodiment, the above-described flow path closing portion 6 is formed by the flow path closing member 32 attached to the tube receiving surface 64 of the container 31. Further, in the present embodiment, the above-described biasing portion 7 is formed by a coil spring as the biasing member 33 that is disposed so as to be elastically deformable between the tube receiving surface 64 of the container 31 and the tube pressing portion 62 of the flow path closing member 32. Furthermore, in the present embodiment, the claw receiving portion as the cap attachment portion 8 described above is formed by a recessed or hole-shaped receiving portion 64a on the tube receiving surface 64 of the container 31 and a protruding portion 61b of the flow path closing member 32 (see FIG. 9).

The container 31 of the present embodiment includes a case portion 11, a film portion 12, and a cover portion 13.

As illustrated in FIGS. 3 and 4, a recess 21 is formed in the case portion 11. The recess 21 of the present embodiment includes a planar bottom surface 21a and a side surface 21b continuously rising from an outer edge portion of the bottom surface 21a. The open end of the recess 21 is formed by an end portion of the side surface 21b opposite to the bottom surface 21a side, that is, an edge portion of the recess 21. The recess 21 of the present embodiment has the above-described shape, but the shape of the recess 21 is not particularly limited. Therefore, the bottom surface 21a of the recess 21 may be, for example, a concave curved surface. Further, the side surface 21b of the recess 21 may have a planar shape or a curved shape.

The film portion 12 has flexibility. As illustrated in FIGS. 3 and 4, the film portion 12 covers the open end of the recess 21 of the case portion 11 and defines the housing space 3a between the recess 21 and the film portion 12. The film portion 12 of the present embodiment is joined to the edge portion of the recess 21. The case portion 11 and the film portion 12 can be joined to each other by, for example, welding or the like, but a joining method is not particularly limited.

The case portion 11 has a fixed shape characteristic not deformed even by the internal pressure of the infusion solution X housed in the housing space 3a. As illustrated in FIGS. 3 and 4, the case portion 11 of the present embodiment is formed by a box body in which the recess 21 is formed and a plate body attached to a side surface side of the box body and in which the tube receiving surface 64 is formed, but is not limited to this configuration. The case portion 11 may be formed by, for example, a single member in which the box body and the plate body described above are integrated, or may be formed by three or more members.

The film portion 12 is deformed by the internal pressure of the infusion solution X housed in the housing space 3a. The thickness of the film portion 12 is smaller than the thickness of the case portion 11 described above. The volume of the housing space 3a varies as the film portion 12 is deformed.

In the present embodiment, the filling port 3c and the ventilation port 3d described above are provided not in the film portion 12 but in the case portion 11.

As described above, the container 31 of the cartridge main body 3 of the present embodiment defines the housing space 3a between the case portion 11 and the film portion 12, but is not limited to this configuration. The container 31 may be formed by, for example, a flexible housing bag that defines the housing space 3a therein, and a case body that covers the periphery of the housing bag.

The cover portion 13 is configured to be openable and closable with respect to the case portion 11. As illustrated in FIGS. 3 and 4, the cover portion 13 includes a cover main body portion 13a and a hinge portion 13b.

The hinge portion 13b is connected to the case portion 11. The cover main body portion 13a is rotatable around the hinge portion 13b between a closed state (see FIGS. 3 and 4) in which the cover main body portion 13a covers the film portion 12 and an open state in which the cover main body portion does not cover the film portion 12.

The cover main body portion 13a and the hinge portion 13b of the present embodiment are formed integrally with the case portion 11, but are not limited to this configuration. That is, the cover portion 13 may be formed separately from the case portion 11 and may be rotatably attached to the case portion 11.

When the housing space 3a is filled with the infusion solution X, the cover main body portion 13a is in an open state not to cover the film portion 12. Therefore, when the housing space 3a is filled with the infusion solution X, even if air bubbles enter the housing space 3a, the film portion 12 can be pushed in from the outside and easily deformed. Therefore, when the housing space 3a is filled with the infusion solution X, the air bubbles in the housing space 3a can be easily moved.

In addition, after the housing space 3a is filled with the infusion solution X, the cover main body portion 13a is in a closed state to cover the film portion 12. Then, the infusion cartridge 2 is attached to the pump main body 1 in a position in which the cover main body portion 13a is in the closed state (see FIGS. 2A to 2C).

In the present embodiment, the tube receiving surface 64 of the cartridge main body 3 is formed by a surface of the container 31 facing the pump main body 1 when attached to the pump main body 1 (see FIG. 1).

The flow path closing member 32 is attached to the tube receiving surface 64 of the container 31. As illustrated in FIGS. 6 and 9, the flow path closing member 32 includes a tube receiving portion 61, a tube pressing portion 62, and a hinge portion 63. The tube receiving portion 61 of the present embodiment includes a plate-like main body portion 83 that receives the tube member 4, a plurality of locking claw portions 61a protruding from the main body portion 83, and a protruding portion 61b protruding from the main body portion 83 and serving as a claw receiving portion on which the locking claw portion 69a of the cap member 5 is caught.

As illustrated in FIG. 6 and the like, a recess 83a is formed in a part of the outer edge of the main body portion 83 of the present embodiment. The tube member 4 is positioned so as to enter the recess 83a. As a result, the tube member 4 is not displaced from a desired position between the tube receiving portion 61 and the tube pressing portion 62. Therefore, the switching between the closed state and the open state of the flow path 4a of the tube member 4 by the tube receiving portion 61 and the tube pressing portion 62 can be executed more stably.

In addition, a through hole 83b is formed in the main body portion 83. As illustrated in FIGS. 11, 13, and the like, a head portion 82 to be described later of the tube pressing portion 62 bent with respect to the tube receiving portion 61 by the hinge portion 63 enters the through hole 83b.

As illustrated in FIGS. 9, 11, 13, and the like, the flow path closing member 32 is attached to the container 31 by the plurality of locking claw portions 61a of the tube receiving portion 61 being inserted into and caught by the recess 64b formed in the tube receiving surface 64 of the container 31.

The tube pressing portion 62 includes an arm portion 81 and a head portion 82. The arm portion 81 is connected to the hinge portion 63. The head portion 82 is connected to a tip end of the arm portion 81, that is, an end of the arm portion 81 opposite to a base end connected to the hinge portion 63.

The tube pressing portion 62 is rotatable with respect to the tube receiving portion 61 with the hinge portion 63 as an axis. That is, as illustrated in FIG. 6, the tube pressing portion 62 is rotatable with respect to the tube receiving portion 61 around a rotation axis of the hinge portion 63 (see a one-dot chain line in FIG. 6). From the state illustrated in FIG. 6, the tube pressing portion 62 is rotated with respect to the tube receiving portion 61 around the hinge portion 63, and the head portion 82 of the tube pressing portion 62 enters the through hole 83b of the tube receiving portion 61. In this state, the flow path closing member 32 is attached to the tube receiving surface 64 of the container 31 (See FIGS. 5B, 11, and 13). As illustrated in FIGS. 11, 13, and the like, the tube member 4 is disposed in a state of being inserted between the tube receiving portion 61 and the arm portion 81 of the tube pressing portion 62.

As described above, the biasing member 33 of the present embodiment is a coil spring. As illustrated in FIGS. 11, 13, and the like, the coil spring as the biasing member 33 is disposed so as to be compressively deformable between the tube receiving surface 64 of the container 31 and the tube pressing portion 62 of the flow path closing member 32.

More specifically, the coil spring as the biasing member 33 of the present embodiment biases the tube pressing portion 62 in a direction in which the tube pressing portion 62 closes the flow path 4a of the tube member 4 between the tube receiving portion 61 and the tube pressing portion 62. With such a configuration, the tube pressing portion 62 can move toward the tube receiving portion 61 and press the tube member 4 in a state in which the tube member 4 is inserted between the tube pressing portion 62 and the tube receiving portion 61 (see FIG. 13).

More specifically, in the present embodiment, the head portion 82 of the tube pressing portion 62 is biased by the biasing member 33. Then, as illustrated in FIG. 13, in a state in which the cap member 5 is detached, the head portion 82 of the tube pressing portion 62 protrudes from the through hole 83b of the tube receiving portion 61 by the biasing force of the biasing member 33. In this state, the tube member 4 is in a closed state by being pressed by the arm portion 81 of the tube pressing portion 62. That is, the position of the tube pressing portion 62 is the closing position, and the flow path closing portion 6 is in the closing position. On the other hand, as illustrated in FIG. 11, in a state in which the cap member 5 is attached, the head portion 82 of the tube pressing portion 62 is pressed by the cap member 5 and housed so as not to protrude from the through hole 83b of the tube receiving portion 61. In this state, the tube member 4 is not pressed by the arm portion 81 of the tube pressing portion 62 and is not closed. That is, the position of the tube pressing portion 62 is the opening position, and the flow path closing portion 6 is in the opening position. As described above, by attachment and detachment of the cap member 5, the tube pressing portion 62 of the present embodiment is movable relative to the tube receiving portion 61 between the closing position (see FIG. 13) that closes the flow path 4a of the tube member 4 between the tube receiving portion 61 and the tube pressing portion 62 and the opening position (see FIG. 11) that does not close the flow path 4a of the tube member 4 between the tube receiving portion 61 and the tube pressing portion 62.

As described above, the cartridge main body 3 of the present embodiment is formed by assembling the container 31, the flow path closing member 32, and the biasing member 33 to each other, but is not limited to this configuration. The cartridge main body 3 may be formed of a single member, or may be formed by assembling two or four or more members. Therefore, in the cartridge main body 3, for example, the container 31 and the flow path closing member 32 described above may be integrally formed.

As illustrated in FIGS. 5A and 5B, the tube member 4 of the present embodiment includes a tube main body 65, a connector 66, and a tube connecting portion 67. The tube main body 65 is a medical tube. The connector 66 is attached to the distal end of the tube main body 65. As illustrated in FIG. 8, the connector 66 of the present embodiment is a locking male connector including a male lure portion 66a and a cylindrical portion 66b surrounding a radially outer periphery of the male lure portion 66a. A female screw portion 66b1 is formed on an inner surface of the cylindrical portion 66b. The inside of the male lure portion 66a communicates with the tube main body 65 in a liquid-tight manner.

The tube connecting portion 67 is attached to the proximal end of the tube main body 65. The tube main body 65 communicates with the housing space 3a in a liquid-tight manner by the tube connecting portion 67. That is, the infusion solution X to be filled in the housing space 3a enters the flow path 4a from the administration port 3e opened to the housing space 3a of the tube connecting portion 67, and is administered into the living body through the flow path 4a. More specifically, the infusion pump 100 illustrated in FIG. 1 can deliver the infusion solution X having entered the flow path 4a of the tube member 4 from the housing space 3a through the administration port 3e to the flow path downstream side by causing peristaltic movement in the tube member 4. On the flow path downstream side of the tube member 4, for example, there is an indwelling needle indwelled in the living body, and the infusion solution X can be administered into the living body through the indwelling needle.

As illustrated in FIGS. 7A to 7C, the cap member 5 includes a cap main body portion 68, an attachment portion 69, and an engagement portion 70.

The cap main body portion 68 is configured to be connectable to the connector 66 of the tube member 4. More specifically, the cap main body portion 68 includes an inner cylindrical portion 68a, an outer cylindrical portion 68b, and a closing wall portion 68c. The outer cylindrical portion 68b covers the radially outer periphery of the inner cylindrical portion 68a. The closing wall portion 68c closes one end sides of the inner cylindrical portion 68a and the outer cylindrical portion 68b. The male lure portion 66a of the connector 66 is internally fitted into the inner cylindrical portion 68a from the open end side opposite to the closing wall portion 68c side of the inner cylindrical portion 68a. In addition, the cylindrical portion 66b of the connector 66 is inserted into an annular space between the inner cylindrical portion 68a and the outer cylindrical portion 68b. In this manner, the cap member 5 of the present embodiment is connected to the connector 66.

The attachment portion 69 is configured to be attachable to the cap attachment portion 8 of the cartridge main body 3. More specifically, the attachment portion 69 of the present embodiment is a locking claw portion 69a that is caught by and engaged with the claw receiving portion serving as the cap attachment portion 8 of the cartridge main body 3. The locking claw portion 69a protrudes from the cap main body portion 68. As illustrated in FIG. 9, the locking claw portion 69a of the present embodiment includes a first claw portion 69a1 and a second claw portion 69a2. The first claw portion 69a1 is caught by the protruding portion 61b of the flow path closing member 32. The second claw portion 69a2 enters and catches a recessed or hole-shaped receiving portion 64a formed on the tube receiving surface 64 of the case portion 11 of the container 31. FIGS. 10 and 11 illustrate a state (Hereinafter, the “cap attached state” may be described.) in which the attachment portion 69 is attached to the cap attachment portion 8 of the cartridge main body 3. In addition, FIGS. 12 and 13 illustrate a state in which the attachment portion 69 is not attached to the cap attachment portion 8 of the cartridge main body 3 (Hereinafter, the “cap detached state” may be described.).

The engagement portion 70 is configured to be engageable with the flow path closing portion 6 in the cap attached state (See FIGS. 10 and 11). More specifically, as illustrated in FIGS. 7B, 7C, 11, and 13, the engagement portion 70 of the present embodiment is formed by a planar pressing surface 70a formed on a part of the outer surface of the outer cylindrical portion 68b of the cap main body portion 68.

As illustrated in FIG. 11, the cap member 5 is attached to the cartridge main body 3 in a state in which the pressing surface 70a as the engagement portion 70 presses the tube pressing portion 62 against the restoring force of the coil spring as the biasing member 33 constituting the biasing portion 7. That is, the tube pressing portion 62 is maintained at the opening position by the cap member 5 in the cap attached state. Therefore, a state in which the flow path 4a of the tube member 4 is not closed is maintained. On the other hand, as illustrated in FIG. 13, the tube pressing portion 62 is biased by the biasing portion 7 in the cap detached state to be at the closing position. That is, the flow path 4a of the tube member 4 is closed.

Next, an operation restricting portion 90 will be described with reference to FIG. 10. The infusion cartridge 2 of the present embodiment includes the operation restricting portion 90. The operation restricting portion 90 restricts the linear pull-out operation of the connector 66 from the cap member 5 in a state in which the cap member 5 is attached to the cartridge main body 3 (see FIG. 10). By providing such an operation restricting portion 90, it is possible to prevent the cap member 5 from being detached from the connector 66 while being attached to the cartridge main body 3. As a result, it is possible to further guide the user to detach the cap member 5 from the cartridge main body 3.

The operation restricting portion 90 of the present embodiment is a restricting wall portion 91. As described above, the connector 66 of the tube member 4 of the present embodiment includes the male lure portion 66a and the cylindrical portion 66b. Further, as described above, the cap member 5 of the present embodiment includes the inner cylindrical portion 68a and the outer cylindrical portion 68b. The connector 66 and the cap member 5 of the tube member 4 are connected to each other by bringing the connector 66 and the cap member 5 close to each other along one central axis O such that all the central axes of the male lure portion 66a, the cylindrical portion 66b, the inner cylindrical portion 68a, and the outer cylindrical portion 68b are aligned with the central axis O. At the time of connection, the male lure portion 66a of the connector 66 is internally fitted into the inner cylindrical portion 68a of the cap member 5. In addition, the cylindrical portion 66b of the connector 66 is inserted between the inner cylindrical portion 68a and the outer cylindrical portion 68b of the cap member 5. Conversely, when the connection between the connector 66 of the tube member 4 and the cap member 5 is released, the connector 66 of the tube member 4 and the cap member 5 may be moved so as to be separated from each other along the central axis O. As described above, the connector 66 of the present embodiment is configured to be attachable to and detachable from the cap member 5 by being moved in the linear attachment/detachment direction C (direction along the central axis O) with respect to the cap member 5.

The cartridge main body 3 of the present embodiment includes the restricting wall portion 91 that restricts the movement of the connector 66 attached to the cap member 5 in the attachment/detachment direction C by abutting against the tube main body 65 or the connector 66 in a state in which the cap member 5 is attached to the cartridge main body 3 (see FIG. 10). That is, in the present embodiment, when the connector 66 is to be removed from the cap member 5 in a state in which the cap member 5 is attached to the cartridge main body 3 (see FIG. 10), the connector 66 or the tube main body 65 connected to the connector 66 abuts against the restricting wall portion 91. As a result, removal of the connector 66 from the cap member 5 is restricted.

As described above, the cartridge main body 3 of the present embodiment includes the tube receiving surface 64. The tube receiving surface 64 supports the flow path closing portion 6 and receives the tube member 4 on the upstream side and the downstream side of the flow path 4a with respect to the flow path closing portion 6. The above-described restricting wall portion 91 of the present embodiment is formed by the tube receiving surface 64. As illustrated in FIG. 10, the central axis O of the present embodiment is inclined with respect to the tube receiving surface 64 in a state in which the cap member 5 is attached to the tube receiving surface 64 of the cartridge main body 3. More specifically, in a state in which the cap member 5 is attached to the tube receiving surface 64 of the cartridge main body 3, the tube main body 65 connected to the connector 66 connected to the cap member 5 is inclined with respect to the tube receiving surface 64 so as to approach the tube receiving surface 64 as being separated from the cap member 5. The inclination angle is preferably 8° to 45°. By setting the angle to 8° or more, it is easy to visually recognize that the connector 66 cannot be detached from the cap member 5. In addition, by setting the angle to 45° or less, it is possible to suppress the tube main body 65 connected to the connector 66 from being excessively bent by the tube receiving surface 64 and generating a kink. From this viewpoint, the inclination angle is more preferably 10° to 30°, and particularly preferably 10° to 25°.

As described above, the restricting wall portion 91 as the operation restricting portion 90 of the present embodiment is formed by the tube receiving surface 64, but is not limited to this configuration. A portion different from the tube receiving surface 64 of the cartridge main body 3 may constitute the restricting wall portion 91.

In addition, the operation restricting portion 90 of the present embodiment is formed by the restricting wall portion 91, but is not limited to this configuration. The operation restricting portion 90 may be realized, for example, by not setting the attachment/detachment direction of the connector 66 and the cap member 5 to the linear attachment/detachment direction C along the central axis O. This type of operation restricting portion 90 may be formed by, for example, a female screw portion provided in the cap member 5 and a male screw portion provided in the connector 66 and capable of being screwed with the female screw portion of the cap member 5. In this manner, the connector 66 and the cap member 5 may be screwed around the central axis O to be connectable, so that the linear pull-out operation along the central axis O of the connector 66 and the cap member 5 may be restricted. That is, the operation restricting portion 90 may be realized by setting the attachment/detachment direction of the connector 66 and the cap member 5 to a direction different from the direction along the central axis O.

Finally, an infusion cartridge 200 as a modification of the infusion cartridge 2 described above will be described with reference to FIG. 14. The infusion cartridge 200 illustrated in FIG. 14 is different from the infusion cartridge 2 described above in that the filling port 3c (see FIG. 1 and the like) is not provided. In the infusion cartridge 200 illustrated in FIG. 14, the housing space 3a is filled with the infusion solution X through the flow path 4a of the tube member 4. Therefore, in the infusion cartridge 200 illustrated in FIG. 14, the flow path 4a of the tube member 4 is preferably opened when the infusion solution X (see FIG. 4) is filled. However, as described above, in consideration of the sterilization treatment at the time of manufacture and the plastic deformation of the tube member 4 from manufacture to use, the flow path 4a of the tube member 4 is preferably not closed before use.

Therefore, the infusion cartridge 200 illustrated in FIG. 14 is configured such that the flow path closing portion 6 is in the closing position in a state in which the cap member 5 is attached to the cartridge main body 3. Therefore, in the infusion cartridge 200 illustrated in FIG. 14, when the cap member 5 is detached from the cartridge main body 3, the position of the flow path closing portion 6 changes from the closing position to the opening position.

Therefore, in the infusion cartridge 200 illustrated in FIG. 14, the cap member 5 is detached in advance from the cartridge main body 3 so that the flow path closing portion 6 maintains the opening position when the infusion cartridge 200 is taken out from the packaging container. Therefore, the user is not guided to the operation of detaching the cap member 5 from the cartridge main body 3. In other words, the user is guided to leave the state in which the cap member 5 is detached from the cartridge main body 3. Therefore, the user can fill the infusion solution X without executing the attaching and detaching operation of the cap member 5 to and from the cartridge main body 3. That is, also in the infusion cartridge 200 illustrated in FIG. 14, it is possible to guide the user so that the operating state of the AFF function becomes a desired state when the infusion cartridge 200 is filled with the infusion solution X.

In the infusion cartridge 200 illustrated in FIG. 14, the cap member 5 may be attached to the cartridge main body 3, for example, after the housing space 3a is filled with the infusion solution X. In this way, it is possible to close the flow path 4a of the tube member 4 after filling with the infusion solution X. As a result, it is possible to prevent the infusion solution X filled in the housing space 3a from flowing back through the flow path 4a of the tube member 4. In addition, because the cap member 5 is attached to the cartridge main body 3, the tube member 4 does not hang down from the cartridge main body 3, and thus the portability of the infusion cartridge 200 filled with the infusion solution X is improved.

In the infusion cartridge 200 illustrated in FIG. 14, configurations other than the above are similar to those of the infusion cartridge 2, and thus the description thereof will be omitted here.

As described above, the cap member 5 only needs to be attachable to the cartridge main body 3 so as to maintain the flow path closing portion 6 in one of the closing position and the opening position. In addition, the flow path closing portion 6 may be configured to change the position from one of the closing position and the opening position to another position in conjunction with the operation of detaching the cap member 5 from the cartridge main body 3.

The flow path closing member 32 and the cap member 5 described above may be distributed as a part of the infusion cartridge 2, 200, or may be distributed as a flow path closing assembly as a part of the infusion cartridge 2, 200. Further, each of the flow path closing member 32 and the cap member 5 may be distributed alone.

The infusion cartridge, the infusion pump, the cap member, and the flow path closing assembly according to the present disclosure are not limited to the specific configurations illustrated in the above-described embodiments and modifications, and various modifications, changes, and combinations are possible without departing from the description of the claims.

The present disclosure relates to an infusion cartridge, an infusion pump, a cap member, and a flow path closing assembly.

What is claimed is:

1. An infusion cartridge comprising:
a cartridge main body that defines a housing space configured to house an infusion solution;
a tube member having a proximal end connected to the cartridge main body and defining a flow path communicating with the housing space; and
a cap member attachable to and detachable from a distal end of the tube member; wherein:
the cartridge main body comprises a flow path closing portion configured to be changed between (i) a closing position in which the flow path closing portion closes the flow path of the tube member and (ii) an opening position in which the flow path closing portion does not close the flow path of the tube member;
the cap member is attachable to the cartridge main body so as to maintain the flow path closing portion in one position among the closing position and the opening position; and
the flow path closing portion is configured to change from said one position to the other position among the closing position and the opening position, in conjunction with an operation of detaching the cap member from the cartridge main body.

2. The infusion cartridge according to claim 1, wherein:
the flow path closing portion comprises:
a tube receiving portion that receives a side surface of the tube member, and
a tube pressing portion movable relative to the tube receiving portion between (i) a closing position in which the tube member is sandwiched between the tube pressing portion and the tube receiving portion to close the flow path and (ii) an opening position in which the tube pressing portion is separated from the tube receiving portion relative to the closing position; and
the flow path closing portion is configured to change between the closing position and the opening position as the tube pressing portion moves relative to the tube receiving portion between the closing position and the opening position.

3. The infusion cartridge according to claim 2, wherein the tube pressing portion is connected to the tube receiving portion via a hinge portion, and is configured to be rotatable with respect to the tube receiving portion with the hinge portion as an axis.

4. The infusion cartridge according to claim 2, wherein the cartridge main body comprises a biasing portion that biases the tube pressing portion.

5. The infusion cartridge according to claim 1, wherein:
the tube member comprises:
a tube main body, and
a connector attached to a distal end of the tube main body and configured to be attachable to and detachable from the cap member; and
the infusion cartridge further comprises an operation restricting portion that restricts a linear pull-out operation of the connector from the cap member in a state in which the cap member is attached to the cartridge main body.

6. The infusion cartridge according to claim 5, wherein:
the connector of the tube member is configured to be attachable to and detachable from the cap member by being moved in a linear attachment/detachment direction with respect to the cap member;
the cartridge main body comprises a restricting wall portion that restricts a movement of the connector attached to the cap member in the attachment/detachment direction by abutting against the tube main body or the connector in a state in which the cap member is attached to the cartridge main body; and
the operation restricting portion is the restricting wall portion.

7. The infusion cartridge according to claim 6, wherein:
the cartridge main body comprises a tube receiving surface that supports the flow path closing portion and receives the tube member on an upstream side and a downstream side of the flow path with respect to the flow path closing portion;
the cap member is attachable to the tube receiving surface of the cartridge main body so as to cover the flow path closing portion; and
the restricting wall portion is formed by the tube receiving surface.

8. The infusion cartridge according to claim 1, wherein:
the cartridge main body includes a filling port configured to allow the housing space to be filled with the infusion solution;
the cap member is attachable to the cartridge main body so as to maintain the flow path closing portion in the opening position; and
the flow path closing portion changes from the opening position to the closing position in conjunction with an operation of detaching the cap member from the cartridge main body.

9. An infusion pump comprising:
the infusion cartridge according to claim 1; and
a pump main body to which the infusion cartridge is attachable.

10. A cap member attachable to and detachable from a distal end of a tube member having a proximal end connected to a cartridge main body that defines a housing space configured to house an infusion solution and comprises a flow path closing portion configured to be changed between (i) a closing position in which the flow path closing portion closes the flow path of the tube member and (ii) an opening position in which the flow path closing portion does not close the flow path of the tube member, wherein:
the cap member is attachable to the cartridge main body so as to maintain the flow path closing portion in one position among the closing position and the opening position; and
the cap member changes a position of the flow path closing portion from said one position to the other position among the closing position and the opening position by being detached from the cartridge main body.

11. A flow path closing assembly comprising:
a cap member attachable to and detachable from a distal end of a tube member having a proximal end connected to a cartridge main body defining a housing space configured to house an infusion solution; and
a flow path closing member attachable to the cartridge main body and configured to change between (i) a closing position in which a flow path of the tube member is closed by the flow path closing member and (ii) an opening position in which the flow path of the tube member is not closed by the flow path closing member; wherein:
the cap member is configured to be attachable to the flow path closing member so as to maintain the flow path closing portion in one position among the closing position and the opening position; and
the cap member changes a position of the flow path closing member from said one position to the other position among the closing position and the opening position by being detached from the flow path closing member.
